(19) United States
(12) Patent Application Publication
(10) Pub. No.: US 2007/0185403 A1
(43) Pub. Date: Aug. 9, 2007

(54) Structure for stabilizing the pressure release of a pressurizing device of a sphygmomanometer

(75) Inventors: Paul Yang, Taipei (TW); Shan-Yi Yu, Taipei (TW); Hsin-Yi Pai, Nantou County (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, CHUNG HO CITY (TW)

(21) Appl. No.: 11/348,252

(22) Filed: Feb. 7, 2006

(51) Int. Cl. A61B 5/02

(52) U.S. Cl. 600/498; 600/490

(57) ABSTRACT

A structure for stabilizing the pressure release of a pressurizing device of a sphygmomanometer includes a valve having an air channel with an end connected to a pressurizing member and another end to an airbag, and the air channel is connected to an interconnecting pipe that is integrally formed with the valve, and the interconnecting pipe includes a pressure release valve having a porous piston body for isolating the air channel. When the pressurizing member is pressurized, the air passes through the air channel to apply a pressure to the airbag. The air in the airbag passes through the air holes of the porous piston body to the outside for measuring blood pressure, and the pulse sensor can detect the pulse signal of the blood pressure more accurately by the airflow.

### BACKGROUND OF THE INVENTION

1. Field of the Invention

The present invention relates to a structure for stabilizing the pressure release of a pressurizing device of a sphygmomanometer, and more particularly to a piston body made of a porous material and having tiny even air holes in the piston body used for stably discharging an airflow in a tiny quantity to measure a pulse signal of blood pressure more accurately.

2. Description of the Related Art

Referring to FIGS. 1 and 2 for a prior art pressurizing device of a sphygmomanometer, the device includes a valve 100 having an interconnected air channel 101, and an end of the valve 100 is connected to a manual pressurization ball 110 and another end is connected to an airbag (not shown in the figure) worn around an arm or a wrist of a user. The valve 100 and the air channel 101 is coupled at their mutually perpendicular end by a screw nut 130, and the screw nut 130 is sheathed with a washer 131, such that when the screw nut 130 is secured onto the valve 100, no gap will be produced. A spring 140 is sheathed into the screw nut 130, and the spring 140 presses against a piston 150. The piston 150 at its periphery includes a washer 151, and a protruding end of the piston 150 is screwed onto a pressure release valve 160. The pressure release valve 160 is movably coupled onto another opening of the valve 100, and the pressure release valve 160 includes a containing space 161 corresponding to the valve 100, and the containing space 161 contains a hollow silicon (rubber) tube 162, and an end of the silicon (rubber) tube 162 is a closed end, and an end opposite to the valve 100 includes an opening, and the surface of the silicon (rubber) tube 162 has a longitudinal crevice 1621 for interconnecting the air in the containing space 161 and the silicon (rubber) tube 162, and the opening end of the silicon (rubber) tube 162 has a screw cylinder 1622, and the screw cylinder 1622 has a penetrating air channel 1623 therein, and the air channel 1623 is interconnected with the opening end of the silicon (rubber) tube 162, and the screw cylinder 1622 can be screwed with a screw nut 1624. The screw nut 1624 is coupled to the containing space 161, so that when the screw nut 1624 is rotated in the screw cylinder 1622, the screw cylinder 1622 originally pressing against the crevice 1621 on the silicon (rubber) tube 162 will be enlarged or reduced accordingly to adjust the size of airflow. When a blood pressure is measured, the manual pressurization ball 110 passes the pressurized air to the airbag through the valve 100 until the air pressure reaches a predetermined maximum setting, the air passing through the valve 100 to the airbag worn around an arm or a wrist will stop. The air in the airbag will pass through the valve 100 and the crevice 1621 of the silicon (rubber) tube 162 in the containing space 161 into the silicon (rubber) tube 162, and the air is discharged to the outside through the air channel 1623 in the screw cylinder 1622 (as shown in FIG. 2). A pulse sensor detects the pulse of high/low blood pressure by the slowly discharging air and sends out the detected high/low blood pressure pulse information for processing, and the measured reading will be displayed on a screen.

Since the silicon (rubber) tube 162 in the pressure release valve 160 is usually made of a silicon (rubber) material, therefore the silicon (rubber) tube 162 will become aged, elastically exhausted, deformed, and brittle after being used for a while, and thus the size of the crevice 1621 on the silicon (rubber) tube 162 will be affected, and the air entering into the containing space 161 no longer maintains a tiny steady flow. As a result, the measurement will not be accurate.

Further, the quantity of discharged air can be adjusted by the screw cylinder 1622 and the screw nut 1624, and the screw threads on the screw cylinder 1622 and the screw nut 1624 are produced by machineries, and thus the precision of the intervals between threads can reach a certain level only, but cannot be adjusted to a very tiny interval accurately. The longitudinal crevice 1621 of the silicon (rubber) tube 162 is cut by a mechanical method, and thus the airflow cannot be discharged steadily in a tiny quantity, and the airflow will not be consistent. The measured air pressure for each measurement cannot be reduced. For example, the airflow entering into the containing space 161 each time is 2˜5 mm Hg/sec, such that we cannot lower the air pressure measured by the pulse sensor (not shown in the figure) per second or shorten the measuring time interval, and thus the measurement cannot be very accurate.

### SUMMARY OF THE INVENTION

In view of the shortcomings of the prior art, the inventor of the present invention aimed at the problem of the prior art sphygmomanometer having an unstable discharged airflow that will cause inaccurate measurements and misjudgments to conduct extensive researches and experiments, and finally invented a structure for stabilizing the pressure release of a pressurizing device of a sphygmomanometer in accordance with the present invention.

Therefore, it is an objective of the present invention to provide a structure for stabilizing the pressure release of a pressurizing device of a sphygmomanometer that includes a valve, and the valve has an air channel, and an end of the air channel is connected to a pressurizing member and another end is connected to an airbag worn around an arm or a wrist of a user, and the air channel is connected to an interconnecting pipe that is extended in a different direction, and the interconnecting pipe and the valve are integrally formed, and the interconnecting pipe includes a pressure release valve having a piston body made of a porous material and movably connected to a piston body that isolates the air channel, such that when the pressurizing device is pressurized, the pressurized air passes through the air channel. The pressurized air can be discharged through the crevice between the pressure release valve and the interconnecting pipe by pressing a press button on the pressure release valve. When a blood pressure is measured, the air in the airbag can pass through the tiny even air holes in the piston body and is discharged steadily to the outside in a low airflow, such that the pulse sensor can accurately detect the pulse signal of the blood pressure due to the tiny steady airflow of the air. Since the piston body will not be exhausted easily, errors of the measured result will not occur due to the unstable airflow.

Another objective of the present invention is to provide a structure for stabilizing the pressure release of a pressurizing device of a sphygmomanometer that includes a piston body made of a precision ceramic material.

### BRIEF DESCRIPTION OF THE DRAWINGS

To make it easier for our examiner to understand the objective, shape, assembly, structure, characteristics and performance of the present invention, the following embodiments accompanied with the related drawings are described in details.

FIG. 1 is a cross-sectional view of a prior art structure;

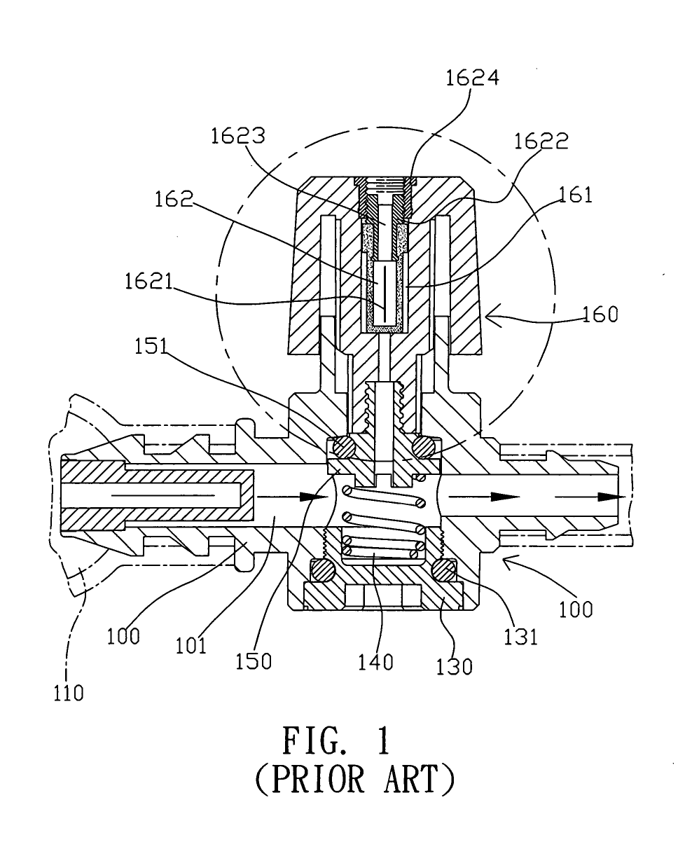

FIG. 2 is an enlarged view of a portion of a prior art device;

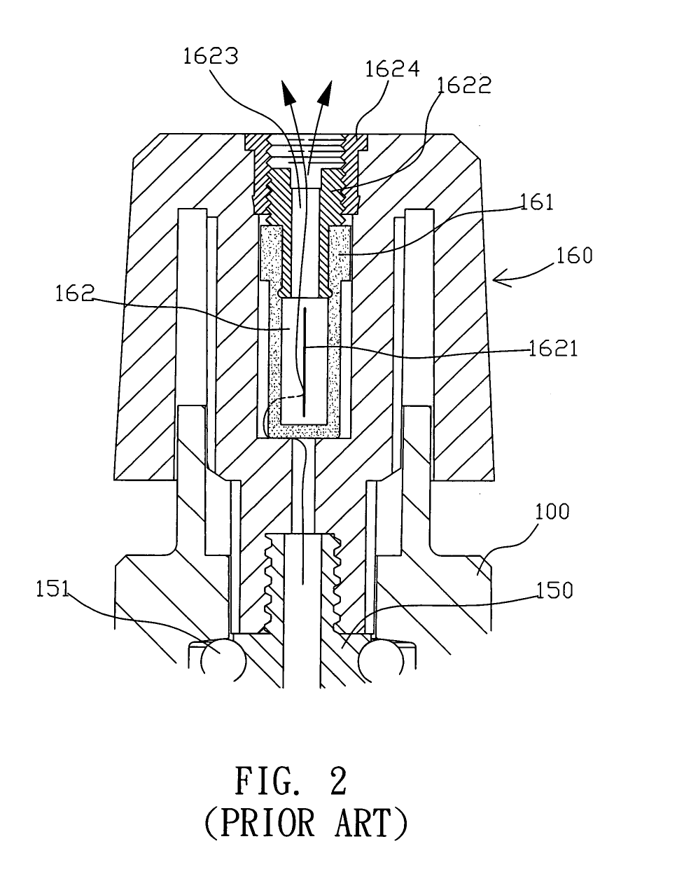

FIG. 3 is a schematic view of a preferred embodiment of the present invention;

FIG. 4 is a perspective view of a valve of the present invention;

FIG. 5 is an exploded view of a valve of the present invention;

FIG. 6 is a cross-sectional view of inflating a valve of the present invention;

FIG. 7 is a cross-sectional view of a valve discharging air quickly according to the present invention; and

FIG. 8 is a cross-sectional view of a valve releasing pressure steadily with a tiny airflow according to the present invention.

### DETAILED DESCRIPTION OF THE PREFERRED EMBODIMENTS

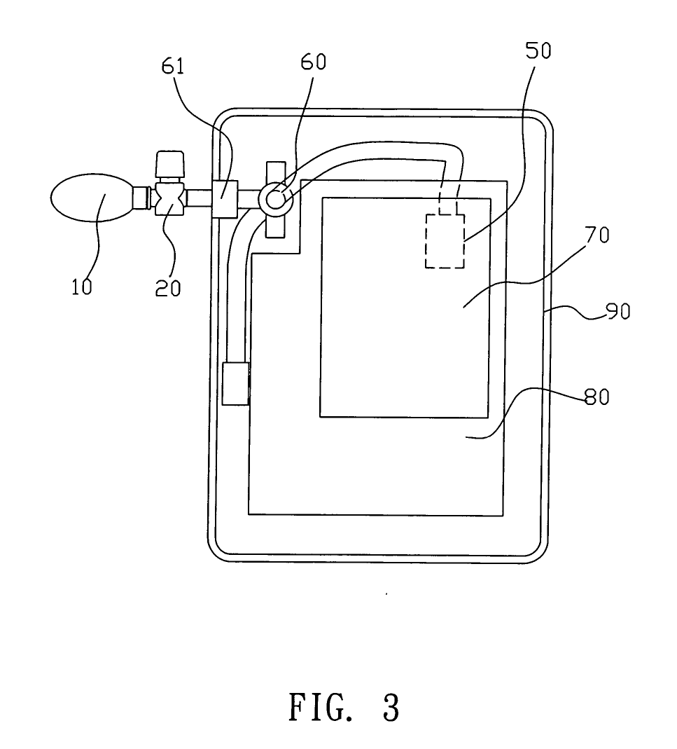

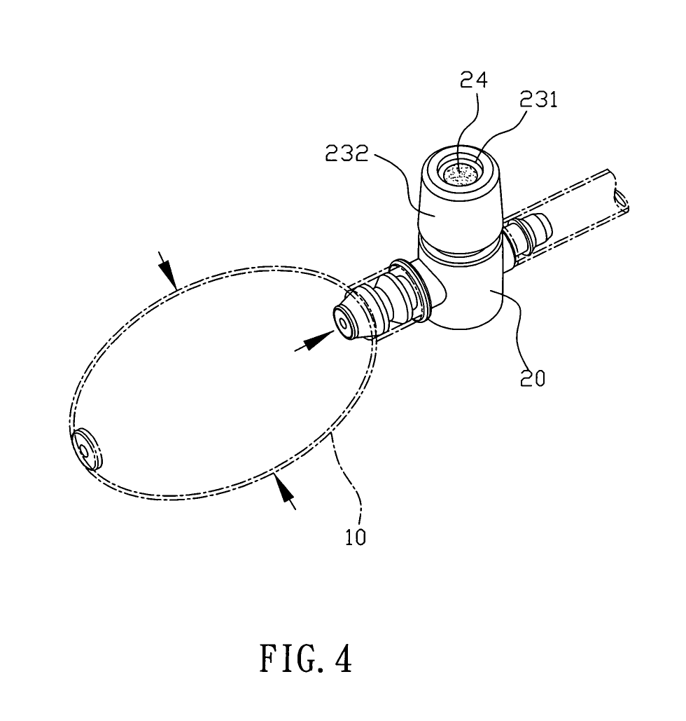

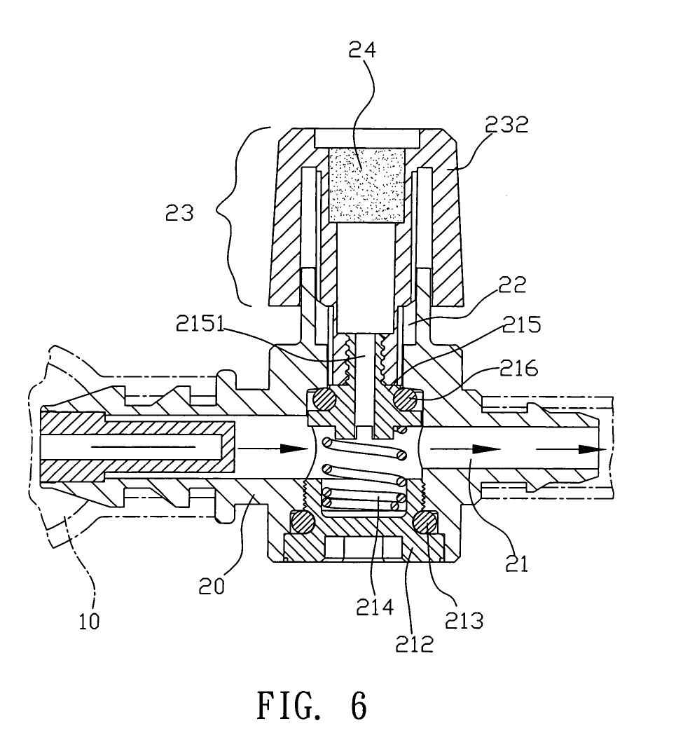

Referring to FIGS. 3 to 5 for the structure for stabilizing the pressure release of a pressurizing device of a sphygmomanometer in accordance with the invention, the sphygmomanometer includes a casing 90, and the casing 90 covers a circuit board 80, and the circuit board 80 is connected to an LCD screen 70 which is exposed from a surface of the casing 90 for displaying the readings of measured high/low blood pressures, and the casing 90 includes a multi-channel interconnecting pipe 60, and the interconnecting pipe 60 has a connector 61 connected to an airbag (not shown in the figure), and the interconnecting pipe 60 is also connected separately to a pulse sensor 50 and a pressurizing device 10, wherein the pulse sensor 50 is installed on the circuit board 80 for detecting pulses of blood pressures to measure a high blood pressure and a low blood pressure. The pressurizing device 10 includes a valve 20 as shown in FIG. 6, and the valve 20 includes an air channel 21, and an end of the air channel 21 has a pressurizing member 10 which is a manual pressurization ball in this preferred embodiment, but the persons skilled in the art can use an electric pump as a substitute, and another end of the air channel 21 passes through the interconnecting pipe 60 to connected with an airbag (not shown in the figure) worn around an arm or a wrist, and the air channel 21 is connected to an interconnecting pipe 22 which is extended in a different direction.

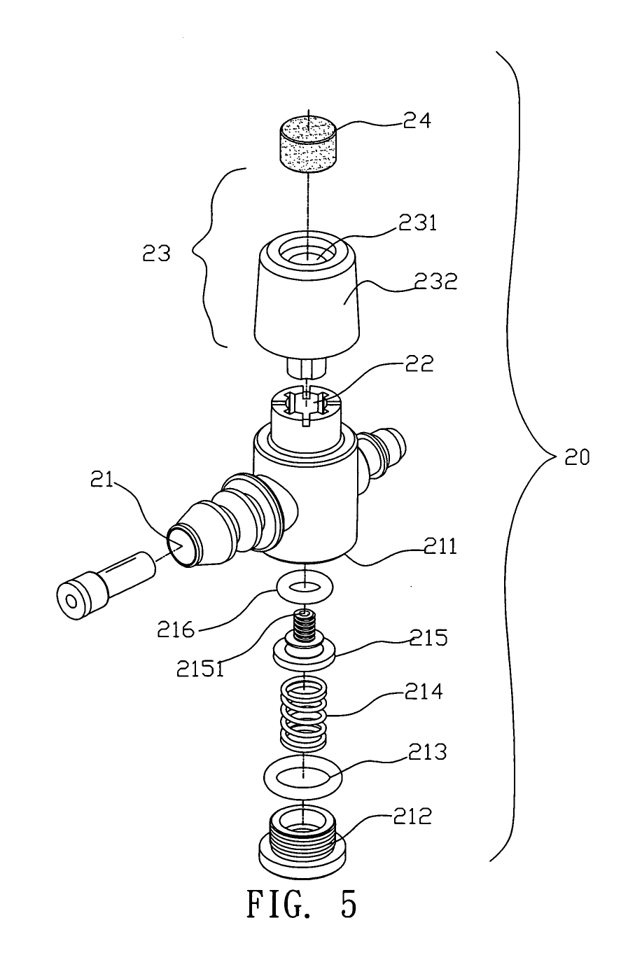

Referring to FIGS. 5 and 6, the interconnecting pipe 22 in the valve 20 includes an opening 211 at a corresponding end, and the opening 211 has a screw nut 212 screwed therein, and the screw nut 212 is sheathed with a washer 213, such that when the screw nut 212 is secured onto the valve 20, no gap will be produced, and the screw nut 212 is also sheathed with a spring 214, and the spring 214 presses against a piston 215, and the piston 215 can move vertically in the air channel 21, and the piston 215 at its periphery is sheathed with a washer 216, and a protruding end of the piston 215 is screwed onto a pressure release valve 23. The pressure release valve 23 includes a containing space 231, and the containing space 231 is interconnected with a through hole 2151 in the piston 215, and the containing space 231 contains a piston body 24 made of a porous material which is precision ceramic in this preferred embodiment, but the persons skilled in the art can use a foam material, a nano material, or similar materials as a substitute. The pressure release valve 23 includes a press button 232 disposed at an end of the piston body 24 away from the piston 215.

Referring to FIG. 6 for the pressurization, the pressurizing member 10 pressurizes air and sends the pressurized air into the air channel 21, so that the pressurized air is passed through the air channel 21 to an airbag (not shown in the figure) worn around an arm or a wrist of a user.

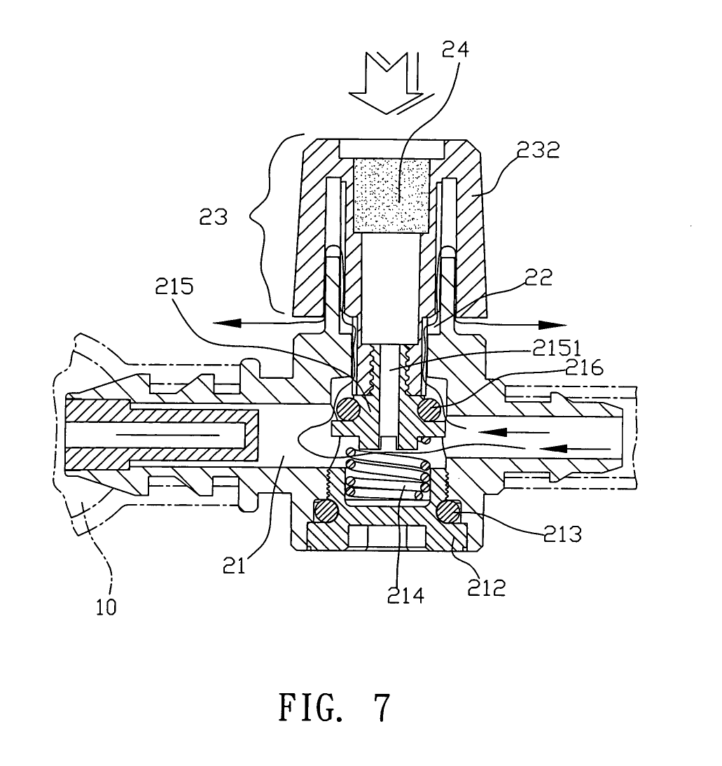

Referring to FIG. 7, for the air in the air bag being discharged rapidly, a press button 232 is pressed, so that the piston 215 leaves its original position, and the air flows quickly from the air gap between the piston 215 and the interconnecting pipe 22 into the interconnecting pipe 22 and flows through the gap between the interconnecting pipe 22 and the pressure release valve 23 to the outside.

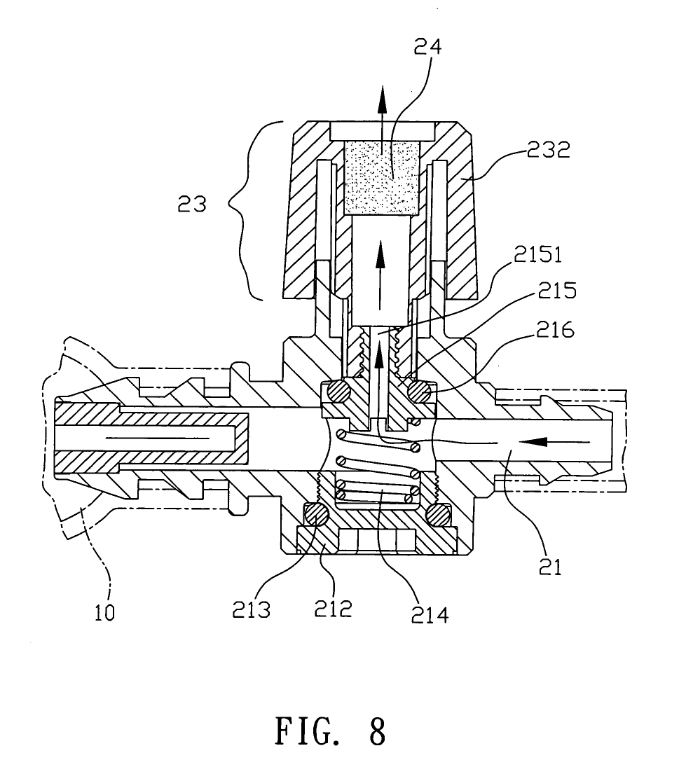

Referring to FIGS. 3 and 8 for measuring a blood pressure, the pressurizing member 10 pressurizes air and sends the pressurized air into the air channel 21, so that the air passes through the air channel 21 to an airbag (not shown in the figure) worn around an arm or a wrist until the air pressure reaches a predetermined maximum setting, and the air in the airbag is passed through the air channel 21 and the through hole 2151 of the piston 215 steadily with a low airflow towards the piston body 24, and then discharged through the tiny even air holes in the piston body 24 to the outside. The pulse sensor 50 can detect a pulse signal of the blood pressure more accurately by the tiny steady airflow. Since the piston body 24 will not be exhausted easily, errors of the measured result will not occur due to unstable airflows. The pulse sensor 50 detects the pulse of a high/low blood pressure by the slowly discharged air, and the detected high/low blood pressure pulse data will be sent to the circuit board 80 for processing, and the measured result will be sent to the LCD screen 70 for its display.

In summation of the above description, the pressurizing device of a sphygmomanometer of the present invention herein enhances the performance and overcomes the shortcoming of the prior art, and further complies with the patent application requirements.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A structure for stabilizing the pressure release of a pressurizing device of a sphygmomanometer, comprising a valve, and said valve having an air channel and an interconnecting pipe interconnected with each other, and said valve having a pressurizing member coupled to an end of said air channel, and another end of said air channel being coupled to an airbag worn around an arm or a wrist of a user, characterized in that: said valve includes a piston body disposed at an end of said interconnecting pipe, and said piston body is made of a porous material, such that when said sphygmomanometer is used, said pressurizing member pressurizes the air and sends the air to said interconnecting pipe, and after said airbag is filled up with air, a blood pressure pulse is measured by the air in said airbag passing through said tiny and even air holes of said piston body and a steady tiny airflow passing through said air holes to release pressure to the outside.

2. The structure for stabilizing the pressure release of a pressurizing device of a sphygmomanometer of claim 1, wherein said piston body is made of precision ceramic.

3. The structure for stabilizing the pressure release of a pressurizing device of a sphygmomanometer of claim 1, wherein said pressurizing member is a manual pressurization ball.

4. The structure for stabilizing the pressure release of a pressurizing device of a sphygmomanometer of claim 1, wherein said pressurizing member is an electric pump.
